(19) United States
(12) Patent Application Publication
(10) Pub. No.: US 2026/0114716 A1
(43) Pub. Date: Apr. 30, 2026

(54) DEVICES, SYSTEMS, AND METHODS FOR USING AN INTEGRAL ADAPTOR TO COUPLE AN ACCESSORY WITH AN INCOMPATIBLE CONNECTOR TO AN ENDOSCOPE

(71) Applicant: EndoSound, Inc., Portland, OR (US)

(72) Inventors: Peter D. HOFFMAN, Portland, OR (US); William J. MCDONOUGH, Portland, OR (US); Kevin Josh COHN, Cornelius, NC (US); Stephen Edward STEINBERG, Boca Raton, FL (US)

(73) Assignee: EndoSound, Inc., Portland, OR (US)

(21) Appl. No.: 19/482,507

(22) PCT Filed: Jun. 7, 2024
(86) PCT No.: PCT/US2024/033140

§ 371 Date: Nov. 7, 2025

Related U.S. Application Data
(60) Provisional application No. 63/507,280, filed on Jun. 9, 2023.

(51) Int. Cl. A61B 1/00; A61M 39/10

(52) U.S. Cl. CPC A61B 1/00128; A61B 1/00137; A61M 39/10; A61M 2039/1077

(57) ABSTRACT

An integral adaptor for connecting an accessory to a medical instrument is disclosed herein. The accessory includes a first connector and the medical instrument includes a second connector. The first connector is incompatible with the second connector. The adaptor can include a first portion configured to selectively engage the first connector of the accessory and a second portion that is integral to the first portion. The second portion is configured to selectively engage the second connector of the medical instrument. The first portion and the second portion collectively define an inner volume that establishes a fluid connection between the accessory and the medical instrument when the accessory is connected to the medical instrument. The adaptor can further include a mechanical connection configured to secure the integral adaptor to the medical instrument when the second portion is selectively engaged with the second connector of the medical instrument.

### CROSS-REFERENCE TO RELATED APPLICATIONS

The present application is related to U.S. Provisional Patent Application No. 63/507,280, titled DEVICES, SYSTEMS, AND METHODS FOR USING AN INTEGRAL ADAPTOR TO COUPLE AN ACCESSORY WITH AN INCOMPATIBLE CONNECTOR TO AN ENDOSCOPE, filed Jun. 9, 2023, the disclosure of which is incorporated by reference in its entirety herein.

### FIELD

The present disclosure is generally related to attachable medical accessories and, more particularly, is directed to devices that can attach a medical accessory to an endoscope instrument in spite of an incompatible connector type.

### SUMMARY

The following summary is provided to facilitate an understanding of some of the innovative features unique to the aspects disclosed herein and is not intended to be a full description. A full appreciation of the various aspects can be gained by taking the entire specification, claims, and abstract as a whole.

In various aspects, an integral adaptor for connecting an accessory to a medical instrument is disclosed. The accessory can include a first connector and the medical instrument includes a second connector. The first connector is incompatible with the second connector. The adaptor can include a first portion configured to selectively engage the first connector of the accessory and a second portion that is integral to the first portion. The second portion is configured to selectively engage the second connector of the medical instrument. The first portion and the second portion collectively define an inner volume that establishes a fluid connection between the accessory and the medical instrument when the accessory is connected to the medical instrument. The adaptor can further include a mechanical connection configured to secure the integral adaptor to the medical instrument when the second portion is selectively engaged with the second connector of the medical instrument.

In various aspects, an integral adaptor for connecting an accessory to a medical instrument is disclosed. The accessory can include a first connector, wherein the medical instrument includes a second connector, and wherein the first connector is incompatible with the second connector, the adaptor including a first portion configured to selectively engage the first connector of the accessory, and a second portion that is integral to the first portion, wherein the second portion is configured to selectively engage the second connector of the medical instrument, wherein the first portion and the second portion collectively define an inner volume that establishes a fluid connection between the accessory and the medical instrument when the accessory is connected to the medical instrument, wherein the integral adaptor is configured to be secured to the medical instrument when the second portion is selectively engaged with the second connector of the medical instrument.

In various aspects, a method of using an integral adaptor to connect an accessory to a medical instrument is disclosed. The accessory can include a first connector and the medical instrument can include a second connector. The first connector is incompatible with the second connector. The method can include causing a first portion of the integral adaptor to selectively engage the first connector of the accessory, causing a second portion of the integral adaptor to selectively engage the second connector of the medical instrument, establishing a fluid connection between the accessory and the medical instrument via an inner volume collectively defined by the first portion and the second portion of the integral adaptor, and securing the integral adaptor to the medical instrument.

These and other features and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

Various features of the aspects described herein are set forth with particularity in the appended claims. The various aspects, however, both as to organization and methods of operation, together with advantages thereof, may be understood in accordance with the following description taken in conjunction with the accompanying drawings as follows:

FIG. 1 illustrates a perspective view of an integral adaptor configured to couple an accessory with an incompatible connector to an endoscope, in accordance with at least one non-limiting aspect of the present disclosure;

FIG. 2 illustrates a side view of another integral adaptor configured to couple an accessory with an incompatible connector to an endoscope, in accordance with at least one non-limiting aspect of the present disclosure;

FIG. 3 illustrates a side view of yet another integral adaptor configured to couple an accessory with an incompatible connector to an endoscope, in accordance with at least one non-limiting aspect of the present disclosure;

FIG. 4 illustrates a flow diagram of a method of using an integral adaptor to couple an accessory with an incompatible connector to an endoscope, in accordance with at least one non-limiting aspect of the present disclosure;

FIG. 5 illustrates a perspective view of yet another integral adapter configured to couple an accessory with an incompatible connector to an endoscope, in accordance with at least one non-limiting aspect of the present disclosure; and

FIG. 6 illustrates a sectioned side view of the integral adaptor of FIG. 5 configured to couple an accessory with an incompatible connector to an endoscope, in accordance with at least one non-limiting aspect of the present disclosure.

Corresponding reference characters indicate corresponding parts throughout the several views. The exemplifications set out herein illustrate various aspects of the invention, in one form, and such exemplifications are not to be construed as limiting the scope of the invention in any manner.

### DETAILED DESCRIPTION

Numerous specific details are set forth to provide a thorough understanding of the overall structure, function, manufacture, and use of the aspects as described in the disclosure and illustrated in the accompanying drawings. Well-known operations, components, and elements have not been described in detail so as not to obscure the aspects described in the specification. The reader will understand that the aspects described and illustrated herein are non-limiting examples, and thus it can be appreciated that the specific structural and functional details disclosed herein may be representative and illustrative. Variations and changes thereto may be made without departing from the scope of the claims. Furthermore, it is to be understood that such terms as “forward”, “rearward”, “left”, “right”, “upwardly”, “downwardly”, and the like are words of convenience and are not to be construed as limiting terms.

It shall be appreciated that medical instruments, such as endoscopes, are produced by a variety of different manufacturers with varying designs. Indeed, even though two medical instruments may be configured for the same or substantially similar medical functions, they may have differing geometries, connections, and other mechanical components that may complicate compatibility. For example, endoscopes produced by two different manufacturers may have different types of connectors at the elongate channel for connecting with accessories, such as a needle. Thus, incompatible connections and interfaces may preclude a clinician from using endoscopes and accessories produced by different manufacturers. Such incompatibility unnecessarily limits a clinician's ability to procure accessories once they have procured an instrument, thereby limiting the clinician's options to use the instrument and decreasing the value of the instrument. This drives costs and unnecessarily limits clinician flexibility. Although several adaptors have been produced to facilitate interoperability of accessories and instruments produced by different manufacturers, these devices generally consist of multiple portions that need to be assembled prior to adapting an instrument for use with an accessory. This is not ideal because it drives additional steps into already complicated and consequential medical situation, introduces more opportunity for human error, and increases the time necessary to setup for a procedure. Additionally, multi-portion adaptors are not well-suited for reuse, because they are harder to sanitize and it is easier to lose a portion, rendering the entire adaptor useless. Accordingly, there is a need for devices, systems, and methods for using an integral adaptor to couple an accessory with an incompatible connector to an endoscope.

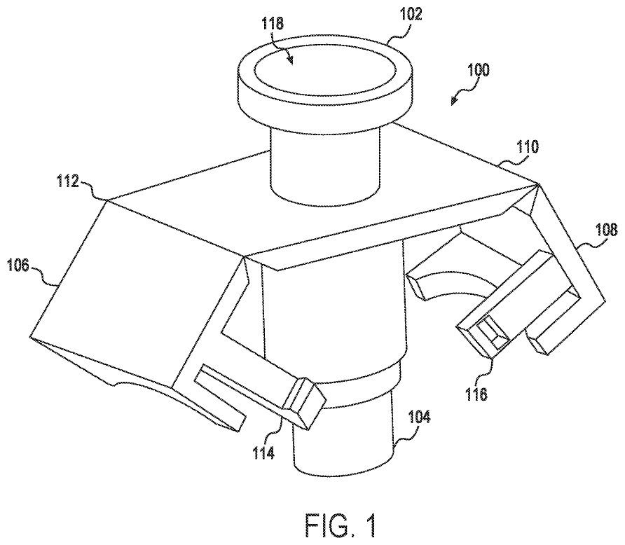

Referring now to FIG. 1, a perspective view of an integral adaptor 100 configured to couple an accessory with an incompatible connector to an endoscope is depicted in accordance with at least one non-limiting aspect of the present disclosure. According to the non-limiting aspect of FIG. 1, the integral adaptor 100 can include a first portion 102 configured for connection to an accessory configured to be selectively attached to a medical instrument, such as an endoscope. For example, according to some non-limiting aspects, the accessory can include a needle. However, it shall be appreciated that the first portion 102 of the integral adaptor 100 of FIG. 1 can be implemented to connect any accessory to the instrument. According to some non-limiting aspects, the first portion 102 can be configured for a luer-type connection or fitting with the accessory. However, according to other non-limiting aspects, the first portion 102 can include other connectors, including a friction fit connector, a small-bore connector, a flange connector, a tapped or threaded connector, and/or a snap or mechanical connector, amongst others.

In further reference to FIG. 1, the integral adaptor 100 can further include a second portion 104 configured to connect to the instrument. According to the non-limiting aspect of FIG. 1, the second portion 104 can be configured to be inserted into a corresponding opening in the instrument. For example, according to some non-limiting aspects, the second portion 104 can include a flange connector configured to establish a fluid connection between the elongate channel of an endoscope, and an inner volume 118 collectively defined by the first portion 102 and the second portion 104 of the integral adaptor 100. This can include a friction fit, for example. However, according to other non-limiting aspects, the second portion 104 can include other connectors, including a luer-type connector, a small-bore connector, a tapped or threaded connector, and/or a snap or mechanical connector, amongst others.

Notably, the connection type of the first portion 102 is different than the connection type of the second portion 104 of the integral adaptor 100 of FIG. 1. The difference between the connection type of the first portion 102 and second portion 104 is intentional and accommodates for the accessory being produced by a different manufacturer than the instrument. Accordingly, it shall be appreciated that the connection types of the first portion 102 and second portion 104 can be altered to accommodate for a particular accessory and/or instrument produced by a particular manufacturer.

Still referring to FIG. 1, the integral adaptor 100 can further include a first side portion 106 and a second side portion 108 configured to be secured about an outer surface of the instrument via a mechanical connection. For example, according to the non-limiting aspect of FIG. 1, the first side portion 106 can include a male connector 114 configured to correspond with and selectively couple to a female connector 116 of the second side portion 108. Of course, the male connector 114 and female connector 116 of the integral adaptor 100 of FIG. 1 are merely illustrative and, according to other non-limiting aspects, the first side portion 106 and second side portion 108 can include any other means of corresponding connectors, as long as they can be selective coupled. It shall be appreciated that, upon coupling the first side portion 106 to the second side portion 108 via the male connector 114 and the female connector 116, the integral adaptor 100 can be securely coupled to the instrument, such that the second portion 104 will remain connected to a corresponding connector of the instrument.

According to the non-limiting aspect of FIG. 1, the first side portion 106 and the second side portion 108 of the integral adaptor 100 can be integral to the material that defines the first portion 102 and the second portion 104 via a first living hinge 110 and second living hinge 112, respectively. For example, the first living hinge 110 and the second living hinge 112 can have a thickness that is less than a thickness of the rest of the integral adaptor 100, thereby forming a thin, flexible hinge or flexure bearing made from the same material as the two rigid pieces, the first portion 102 and the second portion 104, to which the first living hinge 110 and the second living hinge 112 connect. As used herein, it shall be appreciated that the term “living hinge” shall include any hinge that is a made from the same material as the two or more rigid pieces it connects. For example, a living hinge may include a thinner cross section relative to the two or more rigid pieces it connects, thereby allowing more flexibility about the point by which the two or more rigid pieces are connected. However, alternate living hinges, which integrally connect the two or more rigid pieces while facilitating flexibility and motion between the two or more rigid pieces are contemplated by the present disclosure.

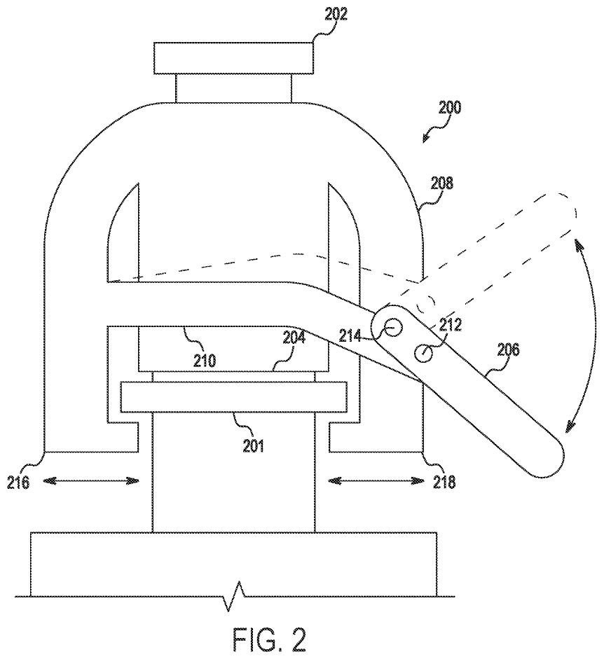

Referring now to FIG. 2, a side view of another integral adaptor 200 configured to couple an accessory with an incompatible connector to an endoscope is depicted in accordance with at least one non-limiting aspect of the present disclosure. Similar to the integral adaptor 100 of FIG. 1, the adaptor 200 of FIG. 2 has a first portion 202 that includes a first connector (e.g., a luer-type connector) configured to selectively attach to an accessory and a second portion 204 that includes a second connector (e.g., a flange connector) configured to selectively attach to an instrument, such as a corresponding flange connector portion 201 of an endoscope. However, according to the non-limiting aspect of FIG. 2, the means by which the integral adaptor 200 is secured to the instrument is different.

For example, the integral adaptor 200 of FIG. 2 can include a first arm 208 and a second arm 210, with a first hinge 214 attached to the first arm 208 and a second hinge 212 attached to the second arm 210 the hinges 212, 214 are configured to pivotally attach a lever 206 between the first arm 208 and the second arm 210. It shall be appreciated that, when the lever 206 is pivoted about the first hinge 214 attached to the first arm 208, the second hinge 212 allows the first lever 206 to apply a force on the second arm 210, which translates through the integral structure and causes a first foot 216 and a second foot 218 positioned at the bottom of a first and second end of the first arm 208 towards and away from the flange connector 201 of the endoscope. In other words, the lever 206 causes the second arm 210 to cause the feet 216, 218 of the first arm 208 to engage and/or disengage the flange connector 201 of the endoscope, thereby selectively securing and releasing the integral adaptor 200 to and from the endoscope.

Although the side view of FIG. 2 illustrates some overlapping arms 208, 210 and hinges 212, 214 of the integral adaptor 200, it shall be appreciated that the first portion 202, the second portion 204, the first arm 208, and the second arm 210 are integral, that is, produced from the same material. Therefore, the only non-integral components of the integral adaptor 200 of FIG. 2 are the hinges 212, 214 and the lever 206, which do not form any part of the core that defines the first portion 202 connector or the second portion 204 connector.

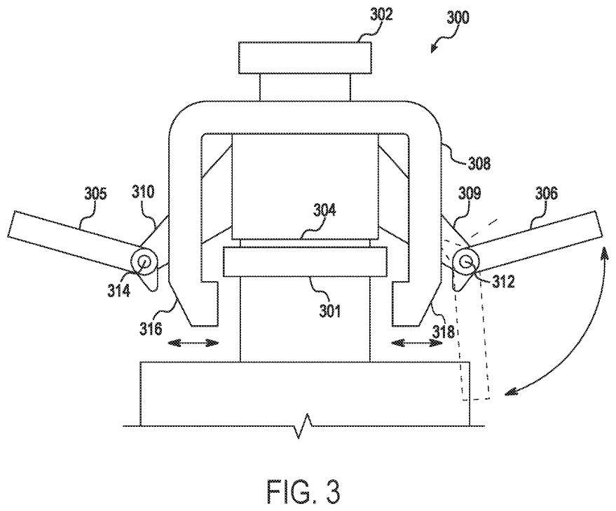

Referring now to FIG. 3, a side view of another integral adaptor 300 configured to couple an accessory with an incompatible connector to an endoscope is depicted in accordance with at least one non-limiting aspect of the present disclosure. Similar to the integral adaptors 100, 200 of FIGS. 1 and 2, the adaptor 300 of FIG. 3 has a first portion 302 that includes a first connector (e.g., a luer-type connector) configured to selectively attach to an accessory and a second portion 304 that includes a second connector (e.g., a flange connector) configured to selectively attach to an instrument, such as a corresponding flange connector portion 301 of an endoscope. However, according to the non-limiting aspects of FIGS. 1 and 2, the means by which the integral adaptor 300 is secured to the instrument is once again different.

For example, the integral adaptor 300 of FIG. 3 can include a first arm 308, a second arm 309, and a third arm 310 all of which can be integrally attached to the core of the integral adaptor 300, which includes and defines the first portion 302 and the second portion 304. A first hinge 314 can attach a first lever 305 to the third arm 310 and second hinge 312 can attach a second lever 306 to the third arm 309. As depicted in FIG. 3, both the first lever 305 and the second lever 306 have an end portion facing the first arm 308 such that, when the first lever 305 and the second lever 306 are pivoted down, the end portion of the first lever 305 and the second lever 306 contacts and pushes on the first arm 308. This causes a first foot 316 and a second foot 318 positioned at the bottom of a first and second end of the first arm 308 towards the flange connector 301 of the endoscope. In other words, the levers 305, 306 cause the feet 316, 318 of the first arm 308 to engage and/or disengage the flange connector 301 of the endoscope, thereby selectively securing and releasing the integral adaptor 300 to and from the endoscope.

Although the side view of FIG. 3 illustrates some overlapping arms 308, 309, 310 and hinges 312, 314 of the integral adaptor 300, it shall be appreciated that the first portion 302, the second portion 304, the first arm 308, the second arm 309, and the third arm 310 are integral, that is, produced from the same material. Therefore, the only non-integral components of the integral adaptor 300 of FIG. 3 are the hinges 312, 314 and the levers 305, 306, which do not form any part of the core that defines the first portion 302 connector or the second portion 304 connector.

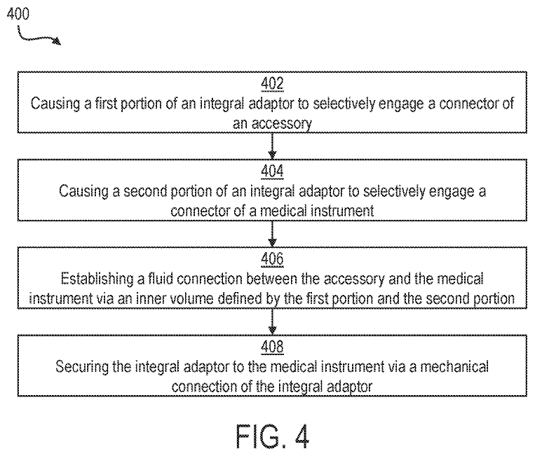

Referring now to FIG. 4, a flow diagram of a method 400 of using an integral adaptor to couple an accessory with an incompatible connector to an endoscope is depicted in accordance with at least one non-limiting aspect of the present disclosure. For example, the accessory can include a first connector and the medical instrument can include a second connector, wherein the first connector is incompatible with the second connector. The method 400 can include causing 402 a first portion of the integral adaptor to selectively engage the first connector of the accessory and causing 404 a second portion of the integral adaptor to selectively engage the second connector of the medical instrument. The method 400 can further include establishing 406 a fluid connection between the accessory and the medical instrument via an inner volume collectively defined by the first portion and the second portion of the integral adaptor. Finally, the method 400 can include securing 408 the integral adaptor to the medical instrument via a mechanical connection of the integral adaptor.

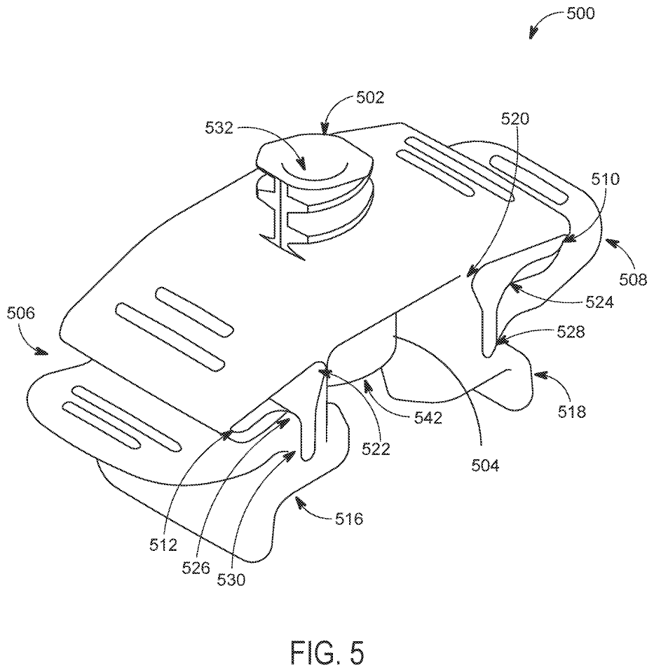

Referring now to FIG. 5, a perspective view of yet another integral adaptor 500 configured to couple an accessory with an incompatible connector to an endoscope is depicted in accordance with at least one non-limiting aspect of the present disclosure. Similar to the integral adaptors 100, 200, 300 of FIGS. 1, 2, and 3, the adaptor 500 of FIG. 5 can have a first portion 502 that includes a first connector (e.g., a luer-type connector) configured to selectively attach to an accessory and a second portion that can include an optional intermediate seal 504. According to the non-limiting aspects of FIG. 5, the optional intermediate seal 504 can be a valve with a thin plastic film with an aperture 542 configured to be in an unbiased, closed position until pressure is added to the aperture 542 to open it. According to another non-limiting aspect of FIG. 5, the optional intermediate seal can be an open ring fluidically coupled to a flange connector of the instrument and configured to enhance the fluidic connection between the integral adaptor 500 and the instrument. The integral adaptor 500 can include an inner volume 532 collectively defined by the first portion 502 and the aperture 542 of the optional intermediate seal 504.. However, according to the non-limiting aspect of FIG. 5, the means by which the integral adaptor 500 is fluidically and mechanically coupled to the instrument is different.

Still referring to FIG. 5, the integral adaptor 500 can include a first side portion 506 and a second side portion 508. The first side portion 506 can be integral to the material that defines the first portion 502 via a first living hinge 512 and a second living hinge 522. According to the non-limiting aspect of FIG. 5, the second side portion 508 can be integral to the material that defines the first portion 502 via a third living hinge 510 and a fourth living hinge 520. For example, the first living hinge 512, second living hinge 522, third living hinge 510, and fourth living hinge 520 can have a thickness that is less than a thickness of the rest of the integral adaptor 500, thereby forming a thin, flexible hinge or flexure bearing made from the same material as the rigid piece, the first portion 502 and, to which the first living hinge 510, second living hinge 522, third living hinge 510, and fourth living hinge 520 connect.

The integral adaptor 500 of FIG. 5 can include a first foot 516 and a second foot 518, the feet 516, 518 configured to selectively attach to an instrument, such as an endoscope. The first foot 516 and the second foot 518 of the integral adaptor can be integral to the material that defines the first side portion 506 and the second side portion 508 via a fifth living hinge 530 and a sixth living hinge 528, respectively. The first foot 516 has an upper radius 536 that is smaller than the lower radius 540. The second foot 518 has an upper radius 534 and a lower radius 538. The difference in the radii facilitates easier installation of the integral adaptor 500 and more difficult for accidental removal when the integral adaptor 500 is mechanically coupled to the instrument. A modulus of elasticity of the living hinges 528, 530 employed by the integral adaptor 500 may be lower than a modulus of elasticity of the first and second side portions 506, 508, thereby imbuing the living hinges with more flexibility and the first and second side portions with more rigidity, relative to each other. The living hinges 528, 530 can made of polypropylene, a durable material that can allow the hinges 528,530 to deform and rotate without breaking, and to withstand the force of the feet 516, 518 coupling to the flange of the instrument. According to the non-limiting aspect of FIG. 5, the modulus of elasticity of the feet 516, 518 can be at least 1.5 GPa and no greater than 8 GPa.

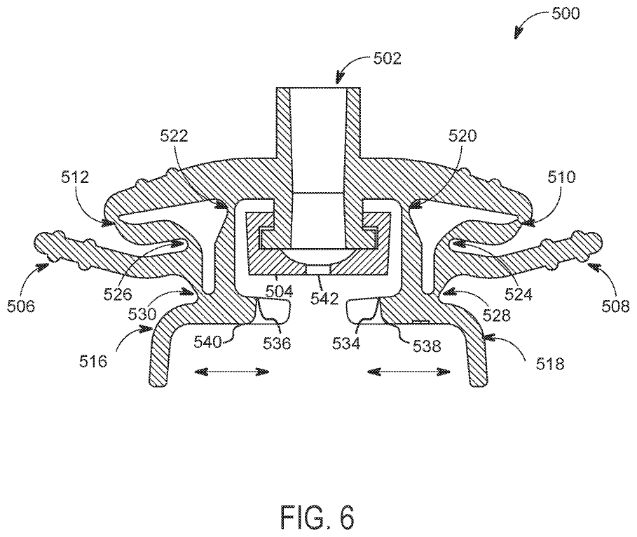

Referring now to FIG. 6, a sectioned side view of the integral adaptor 500 of FIG. 5 is depicted in accordance with at least one non-limiting aspect of the present disclosure. Similar to the integral adaptors 100, 200, 300 of FIGS. 1, 2, and 3, the adaptor 500 of FIG. 5 can have a first portion 502 that includes a first connector (e.g., a luer-type connector) configured to selectively attach to an accessory and a second portion that can include an optional intermediate seal 504. According to the non-limiting aspects of FIG. 6, the optional intermediate seal 504 can be a valve with a thin plastic film with an aperture 542 configured to be in an unbiased, closed position until pressure is added to the aperture 542 to open it. According to another non-limiting aspect of FIG. 6, the optional intermediate seal can be an open ring fluidically coupled to a flange connector of the instrument and configured to enhance the fluidic connection between the integral adaptor 500 and the instrument.

Still referring to FIG. 6, the integral adaptor 500 can include a first foot 516 and a second foot 518, the feet 516, 518 configured to selectively attach to an instrument, such as an endoscope. The first foot 516 and the second foot 518 of the integral adaptor can be integral to the material that defines the first side portion 506 and the second side portion 508 via a fifth living hinge 530 and a sixth living hinge 528, respectively. The second foot 518 has an upper radius 534 and a lower radius 538. The difference in the radii facilitates easier installation of the integral adaptor 500 and more difficult for accidental removal when the integral adaptor 500 is mechanically coupled to the instrument.

For example, the fifth living hinge 530 and sixth living hinge 528 can have a thickness that is less than a thickness of the rest of the integral adaptor 500, thereby forming a thin, flexible hinge or flexure bearing made from the same material as the two rigid pieces, the first side portion 506 and the second side portion 508, to which the fifth hinge 530 and sixth living hinge 528 connect. By using living hinges, the integral adaptor 500 has a higher modulus of elasticity than if the first and second feet, 516, 518 were not integral to the first and second side portions 506, 508.

For example, applying force on the first side portion 506 and second side portion, respectively, translates through the integral structure and causes a first foot 516 and a second foot 518 positioned at the bottom of the first side portion 506 and second side portion 508 to move towards and away from the instrument. In other words, the first side portion 506 and second side portion causes a seventh living hinge 526 and an eighth living hinge 524 to cause the feet 516, 518 to engage and/or disengage the instrument, thereby selectively securing and releasing the integral adaptor 500 to and from the instrument. Applying force on the first foot 516 and the second foot 518 causes the fifth living hinge 530 and the sixth living hinge 528 to cause the feet 516, 518 to engage and/or disengage the instrument, thereby selectively securing and releasing the integral adaptor 500 to and from the instrument. However, it shall be appreciated, that, without applying a force to the living hinges 524, 526, 528, and 530, the integral adaptor remains in a biased condition that keeps the feet 516, 518 positioned relative to the instrument such that the integral adaptor 500 cannot be unintentionally removed from the instrument.

For example, the seventh living hinge 526 and eighth living hinge 524 can have a thickness that is less than a thickness of the rest of the integral adaptor 500, thereby forming a thin, flexible hinge or flexure bearing made from the same material as the two rigid pieces, the first side portion 506 and the second side portion 508, to which the seventh living hinge 526 and eighth living hinge 524 connect. A modulus of elasticity of the living hinges 526, 524 employed by the integral adaptor 500 may be lower than a modulus of elasticity of the first and second side portions 506, 508, thereby imbuing the living hinges with more flexibility and the first and second side portions with more rigidity, relative to each other. The living hinges 528, 530 can made of polypropylene, a durable material that can allow the hinges 528,530 to deform and rotate without breaking, and to withstand the force of the feet 516, 518 coupling to the flange of the instrument. According to the non-limiting aspect of FIG. 5, the modulus of elasticity of the feet 516, 518 can be at least 1.5 GPa and no greater than 8 GPa.

Since the inventive principles of this patent disclosure can be modified in arrangement and detail without departing from the inventive concepts, such changes and modifications are considered to fall within the scope of the following claims. The use of terms such as first and second are for purposes of differentiating different components and do not necessarily imply the presence of more than one component.

Various aspects of the subject matter described herein are set out in the following numbered clauses:
Clause 1. An integral adaptor for connecting an accessory to a medical instrument, wherein the accessory includes a first connector, wherein the medical instrument includes a second connector, and wherein the first connector is incompatible with the second connector, the adaptor including a first portion configured to selectively engage the first connector of the accessory, a second portion that is integral to the first portion, wherein the second portion is configured to selectively engage the second connector of the medical instrument, wherein the first portion and the second portion collectively define an inner volume that establishes a fluid connection between the accessory and the medical instrument when the accessory is connected to the medical instrument, and a mechanical connection configured to secure the integral adaptor to the medical instrument when the second portion is selectively engaged with the second connector of the medical instrument.
Clause 2. The integral adaptor according to clause 1, wherein first connector establishes a luer-type connection between the adaptor and the accessory.
Clause 3. The integral adaptor according to either of clauses 1 or 2, wherein second connector establishes a flange-type connection between the adaptor and the medical instrument.
Clause 4. The integral adaptor according to any of clauses 1-3, further including a first side portion integrally connected to the first portion and the second portion via a first living hinge, wherein the first side portion includes a male connector, and a second side portion integrally connected to the first portion and the second portion via a second living hinge, wherein the second side portion includes a female connector and wherein the mechanical connection includes a selective engagement between the male connector and the female connector.
Clause 5. The integral adaptor according to any of clauses 1-4, further including a first arm that is integral to the first portion and the second portion, a second arm that is integral to the first arm, the first portion, and the second portion, and a lever pivotally connected to a first hinge attached to the first arm and a second hinge attached to the second arm, wherein when the lever is pivoted about the first hinge, the second hinge allows the first lever to apply a force on the second arm, which causes a first foot and a second foot of the first arm to move towards the second connector of the medical instrument, thereby selectively securing the integral adaptor to the medical instrument.
Clause 6. The integral adaptor according to any of clauses 1-5, further including a first arm that is integral to the first portion and the second portion, a second arm that is integral to the first arm, the first portion and the second portion, a third arm that is integral to the first arm, the second arm, the first portion, and the second portion, a first lever pivotally connected to a first hinge attached to the second arm, and a second lever pivotally connected to a second hinge attached to the third arm, wherein when the first lever is pivoted about the first hinge, the first hinge allows the first lever to apply a force on the first arm, which causes a first foot of the first arm to move towards the second connector of the medical instrument, wherein when the second lever is pivoted about the second hinge, the second hinge allows the second lever to apply a force on the first arm, which causes a second foot of the first arm to move towards the second connector of the medical instrument, and wherein the first foot and the second foot selectively secure the integral adaptor to the medical instrument when the first foot and the second foot move towards the second connector of the medical instrument.
Clause 7. The integral adaptor according to any of clauses 1-6, wherein the accessory is a needle.
Clause 8. The integral adaptor according to any of clauses 1-7, wherein the medical instrument is an endoscope.
Clause 9. An integral adaptor for connecting an accessory to a medical instrument, wherein the accessory includes a first connector, wherein the medical instrument includes a second connector, and wherein the first connector is incompatible with the second connector, the adaptor including a first portion configured to selectively engage the first connector of the accessory, and a second portion that is integral to the first portion, wherein the second portion is configured to selectively engage the second connector of the medical instrument, wherein the first portion and the second portion collectively define an inner volume that establishes a fluid connection between the accessory and the medical instrument when the accessory is connected to the medical instrument, wherein the integral adaptor is configured to be secured to the medical instrument when the second portion is selectively engaged with the second connector of the medical instrument.
Clause 10. The integral adaptor according to clause 9, wherein first connector establishes a luer-type connection between the adaptor and the accessory.
Clause 11. The integral adaptor according to either of clauses 9 or 10, wherein second connector establishes a flange-type connection between the adaptor and the medical instrument.
Clause 12. The integral adaptor according to any of clauses 9-11, further including a first side portion integrally connected to the first portion and the second portion via a first living hinge, wherein the first side portion includes a male connector, and a second side portion integrally connected to the first portion and the second portion via a second living hinge, wherein the second side portion includes a female connector and wherein the mechanical connection includes a selective engagement between the male connector and the female connector.
Clause 13. The integral adaptor according to any of clauses 9-12, further including a first arm that is integral to the first portion and the second portion, a second arm that is integral to the first arm, the first portion, and the second portion, and a lever pivotally connected to a first hinge attached to the first arm and a second hinge attached to the second arm, wherein when the lever is pivoted about the first hinge, the second hinge allows the first lever to apply a force on the second arm, which causes a first foot and a second foot of the first arm to move towards the second connector of the medical instrument, thereby selectively securing the integral adaptor to the medical instrument.
Clause 14. The integral adaptor according to any of clauses 9-13, further including a first arm that is integral to the first portion and the second portion, a second arm that is integral to the first arm, the first portion and the second portion, a third arm that is integral to the first arm, the second arm, the first portion, and the second portion, a first lever pivotally connected to a first hinge attached to the second arm, and a second lever pivotally connected to a second hinge attached to the third arm, wherein when the first lever is pivoted about the first hinge, the first hinge allows the first lever to apply a force on the first arm, which causes a first foot of the first arm to move towards the second connector of the medical instrument, wherein when the second lever is pivoted about the second hinge, the second hinge allows the second lever to apply a force on the first arm, which causes a second foot of the first arm to move towards the second connector of the medical instrument, and wherein the first foot and the second foot selectively secure the integral adaptor to the medical instrument when the first foot and the second foot move towards the second connector of the medical instrument.
Clause 15. The integral adaptor according to any of clauses 9-14, wherein the accessory is a needle.
Clause 16. The integral adaptor according to any of clauses 9-15, wherein the medical instrument is an endoscope.
Clause 17. A method of using an integral adaptor to connect an accessory to a medical instrument, wherein the accessory includes a first connector, wherein the medical instrument includes a second connector, and wherein the first connector is incompatible with the second connector, the method including causing a first portion of the integral adaptor to selectively engage the first connector of the accessory, causing a second portion of the integral adaptor to selectively engage the second connector of the medical instrument, establishing a fluid connection between the accessory and the medical instrument via an inner volume collectively defined by the first portion and the second portion of the integral adaptor, and securing the integral adaptor to the medical instrument via a mechanical connection of the integral adaptor.
Clause 18. The method according to clause 17, further including establishing a luer-type connection between the adaptor and the accessory.
Clause 19. The method according to either of clauses 17 or 18, further including establishing a flange-based connection between the adaptor and the medical instrument.
Clause 20. The method according to any of clauses 17-19, further including pivoting a lever about a first hinge such that a second hinge allows a first lever to apply a force that selectively secures the integral adaptor to the medical instrument.

All patents, patent applications, publications, or other disclosure material mentioned herein, are hereby incorporated by reference in their entirety as if each individual reference was expressly incorporated by reference respectively. All references, and any material, or portion thereof, that are said to be incorporated by reference herein are incorporated herein only to the extent that the incorporated material does not conflict with existing definitions, statements, or other disclosure material set forth in this disclosure. As such, and to the extent necessary, the disclosure as set forth herein supersedes any conflicting material incorporated herein by reference and the disclosure expressly set forth in the present application controls.

The present invention has been described with reference to various exemplary and illustrative aspects. The aspects described herein are understood as providing illustrative features of varying detail of various aspects of the disclosed invention; and therefore, unless otherwise specified, it is to be understood that, to the extent possible, one or more features, elements, components, constituents, ingredients, structures, modules, and/or aspects of the disclosed aspects may be combined, separated, interchanged, and/or rearranged with or relative to one or more other features, elements, components, constituents, ingredients, structures, modules, and/or aspects of the disclosed aspects without departing from the scope of the disclosed invention. Accordingly, it will be recognized by persons having ordinary skill in the art that various substitutions, modifications or combinations of any of the exemplary aspects may be made without departing from the scope of the invention. In addition, persons skilled in the art will recognize, or be able to ascertain using no more than routine experimentation, many equivalents to the various aspects of the invention described herein upon review of this specification. Thus, the invention is not limited by the description of the various aspects, but rather by the claims.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although claim recitations are presented in a sequence(s), it should be understood that the various operations may be performed in other orders than those which are described, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

As used herein, the singular form of “a”, “an”, and “the” include the plural references unless the context clearly dictates otherwise.

Directional phrases used herein, such as, for example and without limitation, top, bottom, left, right, lower, upper, front, back, and variations thereof, shall relate to the orientation of the elements shown in the accompanying drawing and are not limiting upon the claims unless otherwise expressly stated.

The terms “about” or “approximately” as used in the present disclosure, unless otherwise specified, means an acceptable error for a particular value as determined by one of ordinary skill in the art, which depends in part on how the value is measured or determined. In certain aspects, the term “about” or “approximately” means within 1, 2, 3, or 4 standard deviations. In certain aspects, the term “about” or “approximately” means within 50%, 200%, 105%, 100%, 9%, 8%, 7%, 6%, 5%, 4%, 3%, 2%, 1%, 0.5%, or 0.05% of a given value or range.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Any numerical range recited herein includes all sub-ranges subsumed within the recited range. For example, a range of “1 to 100” includes all sub-ranges between (and including) the recited minimum value of 1 and the recited maximum value of 100, that is, having a minimum value equal to or greater than 1 and a maximum value equal to or less than 100. Also, all ranges recited herein are inclusive of the end points of the recited ranges. For example, a range of “1 to 100” includes the end points 1 and 100. Any maximum numerical limitation recited in this specification is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, any reference to a processor or microprocessor can be substituted for any “control circuit,” which may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one -application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

1. An integral adaptor for connecting an accessory to a medical instrument, wherein the accessory includes a first connector, wherein the medical instrument includes a second connector, and wherein the first connector is incompatible with the second connector, the adaptor comprising:
a first portion configured to selectively engage the first connector of the accessory;
a second portion that is integral to the first portion, wherein the second portion is configured to selectively engage the second connector of the medical instrument, wherein the first portion and the second portion collectively define an inner volume that establishes a fluid connection between the accessory and the medical instrument when the accessory is connected to the medical instrument;
a mechanical connection configured to secure the integral adaptor to the medical instrument when the second portion is selectively engaged with the second connector of the medical instrument;
a first arm that is integral to the first portion and the second portion;
a second arm that is integral to the first arm, the first portion, and the second portion; and
a lever pivotally connected to a first hinge attached to the first arm and a second hinge attached to the second arm, wherein when the lever is pivoted about the first hinge, the second hinge allows the first lever to apply a force on the second arm, which causes a first foot and a second foot of the first arm to move towards the second connector of the medical instrument, thereby selectively securing the integral adaptor to the medical instrument.

2. The integral adaptor of claim 1, wherein first connector establishes a luer-type connection between the adaptor and the accessory.

3. The integral adaptor of claim 1, wherein second connector establishes a flange-type connection between the adaptor and the medical instrument.

4. (canceled)

5. (canceled)

6. An integral adaptor for connecting an accessory to a medical instrument, wherein the accessory includes a first connector, wherein the medical instrument includes a second connector, and wherein the first connector is incompatible with the second connector, the adaptor comprising:
a first portion configured to selectively engage the first connector of the accessory;
a second portion that is integral to the first portion, wherein the second portion is configured to selectively engage the second connector of the medical instrument, wherein the first portion and the second portion collectively define an inner volume that establishes a fluid connection between the accessory and the medical instrument when the accessory is connected to the medical instrument;
a mechanical connection configured to secure the integral adaptor to the medical instrument when the second portion is selectively engaged with the second connector of the medical instrument;
a first arm that is integral to the first portion and the second portion;
a second arm that is integral to the first arm, the first portion and the second portion;
a third arm that is integral to the first arm, the second arm, the first portion, and the second portion;
a first lever pivotally connected to a first hinge attached to the second arm; and
a second lever pivotally connected to a second hinge attached to the third arm, wherein when the first lever is pivoted about the first hinge, the first hinge allows the first lever to apply a force on the first arm, which causes a first foot of the first arm to move towards the second connector of the medical instrument, wherein when the second lever is pivoted about the second hinge, the second hinge allows the second lever to apply a force on the first arm, which causes a second foot of the first arm to move towards the second connector of the medical instrument, and wherein the first foot and the second foot selectively secure the integral adaptor to the medical instrument when the first foot and the second foot move towards the second connector of the medical instrument.

7. The integral adaptor of claim 1, wherein the accessory is a needle.

8. The integral adaptor of claim 1, wherein the medical instrument is an endoscope.

9. An integral adaptor for connecting an accessory to a medical instrument, wherein the accessory includes a first connector, wherein the medical instrument includes a second connector, and wherein the first connector is incompatible with the second connector, the adaptor comprising:
a first portion configured to selectively engage the first connector of the accessory;
a second portion that is integral to the first portion, wherein the second portion is configured to selectively engage the second connector of the medical instrument, wherein the first portion and the second portion collectively define an inner volume that establishes a fluid connection between the accessory and the medical instrument when the accessory is connected to the medical instrument, wherein the integral adaptor is configured to be secured to the medical instrument when the second portion is selectively engaged with the second connector of the medical instrument;
a first arm that is integral to the first portion and the second portion;
a second arm that is integral to the first arm, the first portion, and the second portion; and
a lever pivotally connected to a first hinge attached to the first arm and a second hinge attached to the second arm, wherein when the lever is pivoted about the first hinge, the second hinge allows the first lever to apply a force on the second arm, which causes a first foot and a second foot of the first arm to move towards the second connector of the medical instrument, thereby selectively securing the integral adaptor to the medical instrument.

10. The integral adaptor of claim 9, wherein first connector establishes a luer-type connection between the adaptor and the accessory.

11. The integral adaptor of claim 9, wherein second connector establishes a flange-type connection between the adaptor and the medical instrument.

12. (canceled)

13. (canceled)

14. An integral adaptor for connecting an accessory to a medical instrument, wherein the accessory includes a first connector, wherein the medical instrument includes a second connector, and wherein the first connector is incompatible with the second connector, the adaptor comprising:
a first portion configured to selectively engage the first connector of the accessory;
a second portion that is integral to the first portion, wherein the second portion is configured to selectively engage the second connector of the medical instrument, wherein the first portion and the second portion collectively define an inner volume that establishes a fluid connection between the accessory and the medical instrument when the accessory is connected to the medical instrument, wherein the integral adaptor is configured to be secured to the medical instrument when the second portion is selectively engaged with the second connector of the medical instrument;
a first arm that is integral to the first portion and the second portion;
a second arm that is integral to the first arm, the first portion and the second portion;
a third arm that is integral to the first arm, the second arm, the first portion, and the second portion;
a first lever pivotally connected to a first hinge attached to the second arm; and
a second lever pivotally connected to a second hinge attached to the third arm, wherein when the first lever is pivoted about the first hinge, the first hinge allows the first lever to apply a force on the first arm, which causes a first foot of the first arm to move towards the second connector of the medical instrument, wherein when the second lever is pivoted about the second hinge, the second hinge allows the second lever to apply a force on the first arm, which causes a second foot of the first arm to move towards the second connector of the medical instrument, and wherein the first foot and the second foot selectively secure the integral adaptor to the medical instrument when the first foot and the second foot move towards the second connector of the medical instrument.

15. The integral adaptor of claim 9, wherein the accessory is a needle.

16. The integral adaptor of claim 9, wherein the medical instrument is an endoscope.

17-20. (canceled)
